(19) United States
(12) Patent Application Publication
(10) Pub. No.: US 2026/0198795 A1
(43) Pub. Date: Jul. 16, 2026

(54) ELECTRICAL IMPEDANCE MAMMOGRAPHY

(71) Applicant: Wei Wang, Brighton Sussex (GB)

(72) Inventor: Wei Wang, Brighton Sussex (GB)

(21) Appl. No.: 19/105,526

(22) PCT Filed: Jun. 23, 2023
(86) PCT No.: PCT/GB2023/051657

§ 371 Date: Feb. 21, 2025

(30) Foreign Application Priority Data
Jun. 23, 2022 (GB) 2209244.9

(51) Int. Cl. A61B 5/0536; A61B 5/00

(52) U.S. Cl. CPC A61B 5/0536; A61B 5/4312; A61B 5/7271

(57) ABSTRACT

Examples relate to a method comprising using a non-target tissue model to virtually purify an experimental electrical impedance signal with respect to target tissue. A method comprising

obtain a purifying impedance transfer function based on an electrical impedance model with one or more non-target tissue components and without a target tissue component; and

purifying an experimental electrical impedance signal using the purifying impedance transfer function to obtain a virtually purified signal for the target tissue component. A method comprising: in dependence upon age and/or a measure of obesity, access an impedance transfer function dependent upon non-tumor tissue; modify an experimental electrical impedance signal using the obtained impedance transfer function to obtain a signal less dependent upon non-tumor tissue and more dependent upon tumor tissue impedance.

### TECHNOLOGICAL FIELD

Embodiments of the present disclosure relate to electrical impedance spectroscopy, electrical impedance imaging, Electrical impedance mammography.

Embodiments of the present invention relate to an apparatus, computer program and method for electrical impedance imaging. In particular, they relate to an apparatus, computer program and method for electrical impedance imaging of the female human breast to facilitate the detection of changes within the breast mass, including changes such as tumors, abnormalities, and malignant changes, such as carcinomas, in the breast.

### BACKGROUND

Electrical impedance mammography (EIM), or Electrical impedance imaging (Ell), also referred to as electrical impedance tomography (EIT) 1 electrical impedance scanner (EIS) and applied potential tomography (APT), is an imaging technique that is particularly used in medical applications.

The technique images the spatial distribution of electrical impedance inside an object, such as the human body. The technique is attractive as a medical diagnostic tool because it is non-invasive and does not use ionizing radiation as in X-ray tomography or the generation of strong, highly uniform magnetic fields as in Magnetic Resonance Imaging (MRI).

Typically a two-dimensional (2D) or three-dimensional (3D) array of evenly spaced electrodes is attached to the object to be imaged about the region of interest (ROI).

Either input voltages are applied across pairs of ‘input’ electrodes and output electric currents are measures at the ‘output’ electrodes or input electric currents are applied between pairs of ‘input’ electrodes and output voltages are measured between at the ‘output’ electrodes or between pairs of output electrodes. For example, when a very small alternating electric current is applied between a pair of ‘input’ electrodes, the potential difference between all other pairs of ‘output’ electrodes is measured. The current is then applied between a different pair of ‘input’ electrodes and the potential difference between all other pairs of ‘output’ electrodes is measured. Image is constructed using an appropriate image reconstruction technique.

Spatial variations revealed in electrical impedance images may result from variations in impedance between healthy and non-healthy tissues, variations in impedance between different tissues and organs or variations in apparent impedance due to anisotropic effects resulting for example from muscle alignment.

Tissue or cellular changes associated with cancer cause significant localized variations in electrical impedance and can be imaged. WO 00/12005 discloses an example of electrical impedance imaging apparatus that can be used to detect breast carcinomas or other carcinoma.

Variations of the individual impedance properties may be used to analyze the structure of an object. For example, in the case of human tissue, variations in the individual impedance properties may be indicative of the presence of an abnormality as this gives rise to electrical characteristics which are different to those exhibited by normal, healthy tissue at the macro level (milli-meter range), or at the micro levels (micro-meter range) showing different special groups of cells, in which will link to abnormality changes in early abnormality changes, particularly malignancy such as carcinoma.

However, the amount of variation of the individual impedance properties either in macro-ranged level or micro-ranged level may be insufficient to enable accurate analysis of the tissue/cellular structures or abnormality either by in-vitro or by in-vivo based EIM technology. For example, the amount of variation of cell membrane capacitance (C) or relaxation frequency (fr) may be insufficient to be readily detectable, for example in images of the object constructed based on those individual impedance properties.

There are therefor advantages to virtually purifying the signals before processing and to be able to identify an abnormal change including benign and malignant lesion.

There are therefor advantages to making sure that the signal is primarily determined by variation in the impedance of the target tissue rather than other tissue to allow to identify benign and malignant changes

### BRIEF SUMMARY

According to various, but not necessarily all, embodiments there is provided examples as claimed in the appended claims.

This is 1st time to use the histo-pathological guided bio-impedance model to purify and identify a cellular based abnormality tissue on 2D,3D EIM imaging by using in-vitro database after “tissue purification”.

### BRIEF DESCRIPTION

Some examples will now be described with reference to the accompanying drawings in which:

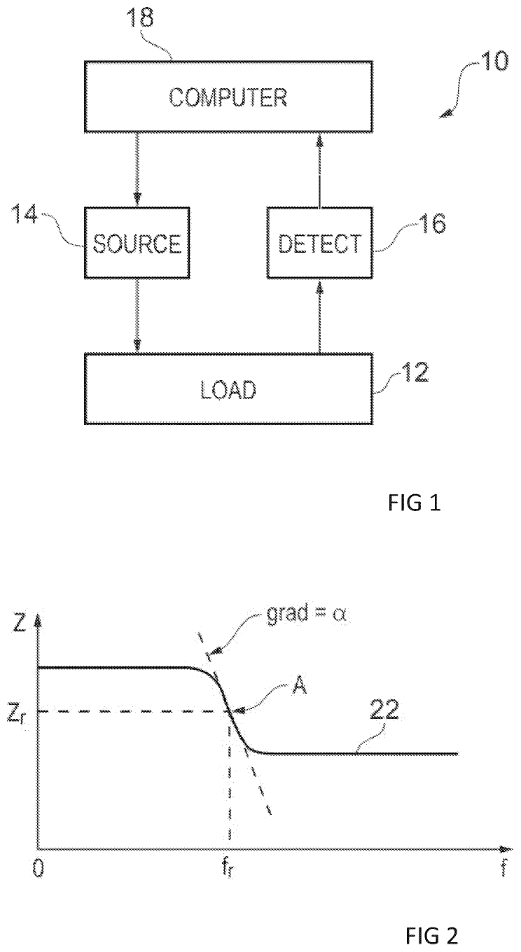

FIG. 1 shows an example of the subject matter described herein;

FIG. 2 shows an example of the subject matter described herein;

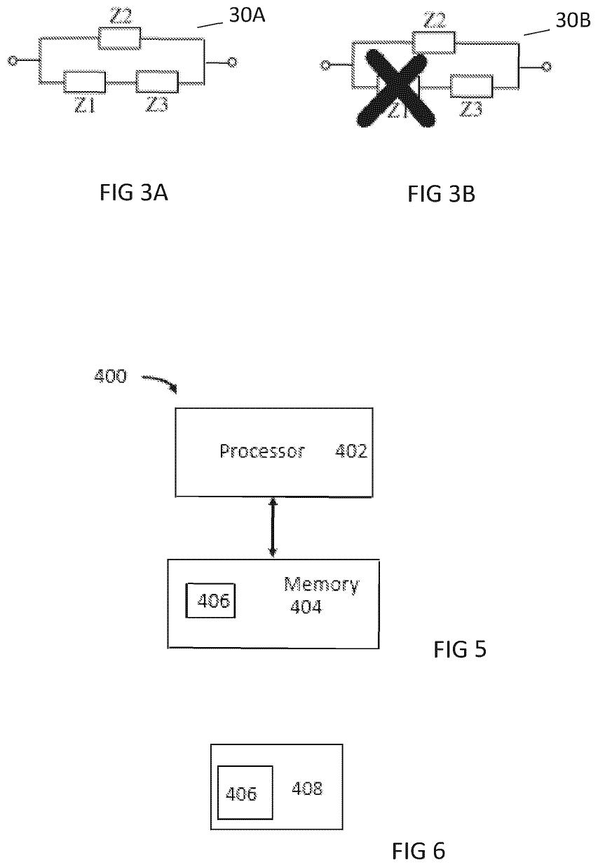

FIG. 3 shows an example of the subject matter described herein;

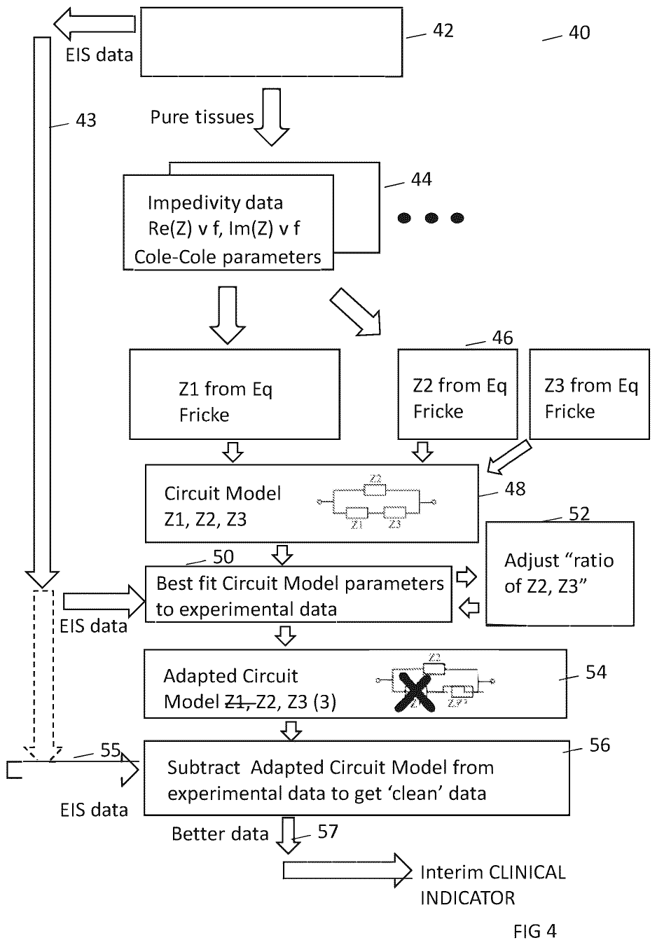

FIG. 4 shows an example of the subject matter described herein;

FIG. 5 shows an example of the subject matter described herein;

FIG. 6 shows an example of the subject matter described herein;

### DETAILED DESCRIPTION

FIG. 1 is a diagrammatic illustration of electrical impedance tomography apparatus;

FIG. 2 show graphs of measured electrical impedance as a function of frequency with single or multiple dispersion;

FIGS. 3A & 3B shows example electrical impedance circuit models of an object; and

FIG. 4 shows a flowchart.

FIG. 5 shows a controller & FIG. 6 show a computer program.

FIG. 1 illustrates diagrammatically electrical impedance measurement or electrical impedance tomography (EIT) apparatus 10 for measuring impedance data for a load 12. The load 12 comprises an electrically conductive object to which are attached a plurality of electrodes. The term ‘electrically conductive’ means that the object is capable of conducting an electric current but it does not necessarily need to conduct current very well. The apparatus 10 further comprises a signal source 14, a signal detector 16 and a computer 18. In one embodiment, the signal source provides, as an input signal, an electric current and the signal detector detects, as an output signal, voltage. In another embodiment, the signal source provides, as an input signal, a voltage and the signal detector detect, as an output signal, electric current.

The computer typically comprises at least a processor and a memory. The memory stores a computer program which when loaded into the processor controls the computer.

The input signal is applied using the source 14 to the object via electrodes and the resulting output signals present at same or other electrodes are measured using the detector 16. This process is repeated for different frequencies of input signal. For example, the electric signal may be applied by the signal source 14 at a number of frequencies between 0 Hz (direct current) and 100 MHz, to enable frequency dependent electrical impedance data to be obtained for the object.

The separation of the electrodes used for the impedance measurements determines the resolution or scale at which the object is analyzed. The electrical impedance measurements may be obtained at an expected scale of interest (e.g., micro-meter or millimeter range). As an example of the scale of interest, for a biological object, we may be interested in the single cell or in the group cell level or at tissue or histology level, such as lobule or duct in breast tissue. Subsequently the obtained electrical impedance data will be analyzed using a transfer function of an assumed electrical model to determine a plurality of electrical impedance properties for the object. The electrical model used may depend upon the resolution/scale of the impedance measurements.

Referring to FIG. 2, the electrical impedance data obtained using the above method can be plotted as a function of frequency. This plot 22 represents the impedance changes vs frequencies or transfer function for the object. The computer 18 is operable to execute an appropriate algorithm to analyze the obtained impedance transfer function or frequency dependent impedance properties and thereby determine a plurality of electrical impedance properties for the object. The electrical impedance properties typically include one or more of:
a) the impedance at the limit ω->0 (lower limit)
b) the impedance at the limit ω->∞ (upper limit)
c) (i) the relaxation frequency at which there is a change in the tissue structure, membrane or cellular related impedance properties
   (ii) the impedance at that change frequency
   (iii) the gradient of the change of impedance, particularly at the relaxation frequencies relating to cellular membranes' property and the distribution of diversity of the dedicated the group of cell membranes

For example, if there are N dispersions including the Alpha, Beta and Gamma dispersions of biological materials [Cole K S, Permeability and impermeability of cell membranes for ions. Cold Spring Harbor Symp. Quant. Biol. 8 pp110-22, 1940] within the frequency range used, where N>1, then the dispersion frequencies ω₁, ω₂, . . . ω_{N-1}, ω_{N}, are identified and the electrical impedance properties for a particular dispersion m would typically include one or more of:
a) For m=1, the impedance at the lower (global) limit ω->0
   For m>1, the impedance at the lower (local) limit ω->ωₘ-a, where a< (ωₘ-ωₘ₋₁) and may possibly be ½ (ωₘ-ωₘ₋₁)
b) For m=N, the impedance at the upper (global) limit ω->∞

For m<N, the impedance at the upper (local) limit ω->ωₘ+b, where b<(ωₘ₊₁-ωₘ) and may possibly be b ~½ (ωₘ₊₁-ωₘ)
c) (i) the relaxation frequency ωₘ (fᵣₘ) at which there is a change in the impedance
   (ii) the impedance at that change frequency
   (iii) the gradient of the change

The amount of variation of one or more of these impedance properties can be used to analyze the structure of the object due to the intra/extra cellular or intra/extra cellular-like related changes in either macro-range or micro-range levels' pathology property.

In some embodiments, the object under analysis is modeled using an equivalent electrical impedance circuit. The object may be modeled using an equivalent electrical impedance circuit 20 illustrated in FIG. 3. Objects which may be modeled using the equivalent electrical impedance circuit 20 may, in a non-limiting example, include human or animal tissue, and porous or other cellular or cellular-like materials, in cascaded structures, such as each of Z1, Z2, Z3 would contain similar 3-element (like Z1-1, Z1-2, Z1-3 etc to be able to represent single-cell, small group cell at micro-range or large group cell at macro or tissue level . . . ).

In the illustrated embodiment, the equivalent electrical impedance circuit 20 comprises a cell portion 21 in parallel with an extra-cell portion 23. The cell portion 21 has a capacitance C and a resistance Rᵢ in series. The resistance C is associated with the cell membrane/boundary and the resistance Rᵢ, is associated with the interior of the cell. The extra-cell portion 23 has a resistance Rₑ. The resistance Rₑ is associated with the structure outside the cell. The resistance Rₑ is connected in parallel with the series connected capacitance C and resistance Rᵢ.

A non-limiting example of a single dispersion impedance transfer function for this circuit is:

$$Z ( ω ) = \frac{Re ( 1 + j \cdot ω \cdot C \cdot Ri )}{1 + j \cdot ω \cdot C \cdot ( Re + Ri )}$$

In the limit ω->0, Z->Rₑ

In the limit ω->∞, Z->Rᵢ//Rₑ i.e. R Rₑ/(Rᵢ+Rₑ)

There is a change (dispersion) at frequency fr and an impedance Zr that has a gradient α.

The transfer model for multiple dispersion in biological tissue can be modeled by the Cole-Cole equation (Cole K S 1940, Cole K S 1941, McAdams E T et al, 1995) as follows:

$$Z = R ∝ + ( R 0 - R ∝ ) / ( 1 + ( jf / fr ) ) ( 1 - α )$$

Usually this equation can be rewritten as the equation below if a three-element electrical equivalent circuit is used for a simple modeling cell suspensions (Fricke and Morse, 1925) or tissues:

$$Z = {R}_{e} \cdot {R}_{i} / ( {R}_{e} + {R}_{i} ) + ( {R}_{e} - {R}_{e} \cdot {R}_{i} / ( {R}_{e} + {R}_{i} ) ) / ( 1 + ( jf / fr ) ) ( 1 - α )$$

Where R_{oc} is the result of paralleling Rₑ and Rᵢ.

There are changes (dispersion) at frequency fᵣᵢ and impedance Zᵣᵢ that has a gradient.

As indicated above, the computer 18 is operable to execute an appropriate algorithm to analyze the measured impedance data and extract a plurality of electrical impedance properties for the object under analysis. For example, based on the measured impedance data, the algorithm may be operable to plot impedance data points as a function of frequency and produce a best fit line 22 using the model to form the transfer function illustrated in FIG. 2. From this transfer function, the computer 18 is capable of determining a plurality of individual impedance properties for the object. These impedance properties may include:
a) the impedance at the limit ω->0, which gives Rₑ
b) the impedance at the limit ω->∞, which gives Rᵢ Rₑ/(Rᵢ+Rₑ)
c) (i) the relaxation frequency fr at which there is a change in the impedance
   (ii) the impedance Z, of the transfer function at that change frequency
   (iii) the gradient α of the change which gives the relaxation factor.

The impedance properties may be used to determine further impedance properties using the model.

For example, if both Rₑ and Rᵢ Rₑ/(Rᵢ+Rₑ) are known then Rᵢ can be determined.

The impedance Zᵣ of the transfer function at the change (dispersion) frequency fᵣ, is where the capacitor dominates the transfer characteristic as with each small increases in frequency it conducts significantly better reducing the impedance. The impedance Zᵣ at the change (dispersion) frequency fᵣ, can be modelled as 1/(j.2π fᵣ.C). Therefore C can be determined as 1/(j.2π fᵣ. Zᵣ).

Variations of the individual impedance properties (Rₑ, Rᵢ, fᵣ, Zᵣ, α, C) may be used to analyze the structure of an object at certain level either at macro or micro range. For example, in the case of human tissue, variations in the individual impedance properties may be indicative of the presence of an abnormality as this gives rise to electrical characteristics which are different to those exhibited by normal, healthy tissue.

However, the amount of variation of the individual impedance properties may be insufficient to enable accurate analysis of the structure. For example, the amount of variation of cell membrane capacitance (C) or relaxation frequency (fᵣ) may be insufficient to be readily detectable, for example in images of the object constructed based on those individual impedance properties.

FIG. 3A illustrates a mixed impedance model of the object under analysis for representing a reality different way of mixed tissues including normal stoma, glandular or abnormal tissue growth. In the illustrated embodiment, the equivalent electrical impedance circuit 30A comprises an inclusion portion 31 in parallel with an inter-inclusion portion 33. The inclusion portion 31 has impedance Z1 and impedance Z3 in series. The inter-inclusion portion 33 has impedance Z3. The impedance Z3 is associated with the structure outside the inclusion. The impedance Z3 is connected in parallel with the series connected impedance Z1 and Z2.

The impedance transfer function for this circuit 30A is:

$${Z}_{A} ( ω ) = \frac{Z 1 \cdot Z 2 \cdot Z 3}{Z 1 \cdot Z 2 + Z 1 \cdot Z 3 + Z 2 \cdot Z 3}$$

In this model, Z1 is the impedance of the target tissue. That is, the tissue that is targeted for detection.

FIG. 3B illustrates a mixed impedance model of the object under analysis in the limit of the impedance of the target tissue being zero. In the illustrated embodiment, the equivalent electrical impedance circuit 30b comprises impedance Z3 in parallel with the impedance Z2.

The impedance transfer function for this circuit 30B is:

$${Z}_{B} ( ω ) = \frac{Z 2 \cdot Z 3}{Z 2 + Z 3}$$

$$Or$$

$${Z}_{B} ( ω ) = \frac{R \cdot Z 2 \cdot Z 3}{R \cdot ( Z 2 + Z 3 )}$$

R is an optimization factor, and the impedance Z2 is a reference impedance for first non-targeted tissue and the impedance Z3 is a reference impedance for second non-targeted tissue that is different to the first non-targeted tissue.

### Let

S be experimental data (in frequency domain);
Z_{A} be a model transfer function that includes the target tissue and non-target tissues it is based on a circuit model that includes the electrical impedance of the target tissue and the electrical impedances of the non-target tissues e.g., Z1, Z2, Z3.
Z_{B} be a model transfer function for the non-target tissues based it is based on the same circuit model but which now includes the electrical impedance of the non-target tissues e.g., Z2, Z3 and doe not include the electrical impedance of the target tissue Z1.
N be the noise signal that is the signal arising from the non-target tissue
C be the desired signal (the experimental signal S with noise N removed)

$$S = {Z}_{A} * s \qquad [ Eq 1 ]$$

$$⇒ s = {Z}_{A}^{- 1} * S$$

$$N = {Z}_{B} * s$$

$$Substituting Eq 1$$

$$N = {Z}_{B} * {Z}_{A}^{- 1} * S$$

$$C = S - N = S - {Z}_{B} * {Z}_{A}^{- 1} * S$$

Therefore, C can be determined from S and knowledge of Z_{B}, Z_{A}.
S(t) be experimental data (in time domain);
S be experimental data (in frequency domain);
Z_{A} be a model transfer function that includes the target tissue and non-target tissues it is based on a circuit model that includes the electrical impedance of the target tissue and the electrical impedances of the non-target tissues e.g. Z1, Z2, Z3.
Z_{B} be a model transfer function for the non-target tissues based it is based on the same circuit model but which now includes the electrical impedance of the non-target tissues e.g. Z2, Z3 and doe not include the electrical impedance of the target tissue Z1.
N be the noise signal that is the signal arising from the non-target tissue in frequency domain, and N (t) be the equivalent signa in the time domain.
C be the desired signal (the experimental signal S with noise N removed) in frequency domain, and C(t) be the equivalent signa in the time-space domain.

$$S = {Z}_{A} * s \qquad [ Eq 1 ]$$

$$⇒ s = {Z}_{A}^{- 1} * S$$

$$N = {Z}_{B} * s$$

$$Substituting Eq 1$$

$$N = {Z}_{B} * {Z}_{A}^{- 1} * S$$

$$N ( t ) = {T}^{- 1} \{ {Z}_{B} * {Z}_{A}^{- 1} * T \{ S ( t ) \} \}$$

$$C ( t ) = S ( t ) - N ( t ) = S ( t ) - {T}^{1} \{ {Z}_{B} * {Z}_{A}^{- 1} * T \{ S ( t ) \} \}$$

Where T { } is the forward transform from time-space to frequency and T⁻¹{ } is the reverse transform from frequency to time-space.

Therefore C(t) can be determined from S(t) and knowledge of Z_{B}, Z_{A}.

FIG. 4 illustrates a method comprising using a non-target tissue model (e.g FIG. 3B) to virtually purify an experimental electrical impedance signal with respect to target tissue. In some examples, the target tissue is tumor tissue.

The method illustrated comprises:
obtaining a purifying impedance transfer function (Z_{B}) based on an electrical impedance model (FIG. 3B) with one or more non-target tissue components (Z2, Z3) and without a target tissue component (Z1);
purifying an experimental electrical impedance signal (S, S(t)) using the purifying impedance transfer function (Z_{B}) to obtain a virtually purified signal (C, C(t)) for the target tissue component.

Purifying an experimental electrical impedance signal (S, S(t)) using the purifying impedance transfer function (Z_{B}) to obtain a virtually purified signal for the target tissue component can be performed by assuming an electrical impedance model (FIG. 3A) comprising the target tissue component and the one or more non-target tissue components, wherein the target tissue is tumor tissue and the non-target tissue is non-tumor tissue.

The transfer function (Z_{A}) of the full electrical impedance model (FIG. 3A) can be used with a transfer function (Z_{B}) of the reduced electrical impedance model (FIG. 3B) to obtain a virtually purified signal (C, C(t)) for the target tissue component.

The a transfer function (Z_{A}) of the full electrical impedance model (FIG. 3A) is based on target tissue impedance Z1 and non-target tissue impedances Z2, Z3. The reduced electrical impedance model (FIG. 3B) is based on non-target tissue impedances Z2, Z3.

The non-target tissue impedances Z2, Z3 can be based on reference impedance values for the pure non-target tissue modified by an optimization factor (R).

The optimization factor (R) correlates to one or more characteristics of the patient such as age and/or obesity/(different BMI)

The assignment of an appropriate optimization factor (R) to a subjects a classification problem that can be addressed using machine learning.

In at least some examples, the method comprises:
in dependence upon age and/or a measure of obesity (or BMI), access an impedance transfer function Z_{B} dependent upon non-tumor tissue (e.g., dependent upon Z2, Z3, R);
modify an experimental electrical impedance signal using the obtained impedance transfer function Z_{B} to obtain a signal less dependent upon non-tumor tissue impedance (Z1, Z2) and more dependent upon tumor tissue impedance (Z1).

One measure of obesity is body-mass-index

The non tumor tissue impedance can be based on connective tissue impedance and adipose tissue impedance.

The tumor tissue impedance can be a benign, such as fibroadenoma tissue impedance or malignant, such as ductal carcinoma tissue, or lobular carcinoma tissue or ductal carcinoma in-situ (DCIS) impedance depending on target.

The experimental signal can be an electrical impedance signal from an in vitro measurement. This can be used to analyze an in vitro sample.

The experimental signal can be an electrical impedance signal from an in vivo measurement. This can be used to analyze a live subject.

The electrical impedance signal could be from a 2D or 3D region of interest (ROI) on EIM image with known tissue areas at the ROI, based either on tissue-impedance database or measured BMI from this specific patient of volunteer.

The electrical impedance signals can be used for electrical impedance imaging.

In at least some examples, the electrical impedance model is a mixed impedance model which includes a target tissue impedance (Z1) and non-target tissue impedance (Z2, Z3).

The target tissue impedance Z1 and a first non-target tissue impedance Z3 are in electrical series as a combination, and that combination is in electrical parallel to a second non-target tissue impedance Z2-see FIG. 3A.

The target tissue impedance Z1 can be one of a fibroadenoma reference impedance or inductal carcinoma reference impedance depending upon a desired target.

The first non-target tissue impedance Z3 is one of connective tissue impedance and adipose tissue impedance and the second non-target tissue impedance Z2 is the other one of connective tissue impedance and adipose tissue impedance.

The method can use in vitro samples to determine the reference impedance of target tissue (Z1) and/or use in vitro samples to determine the reference impedance of non-target tissue. It can use an in vitro sample to determine the first non-target tissue reference impedance Z3. It can use an in vitro sample to determine the second non-target tissue reference impedance Z2.

The electrical impedance model can be determined by best fit to in vitro data for one or more samples. For example, a relative ratio R between first non-target tissue reference impedance Z3 and second non-target tissue reference impedance for the electrical impedance model is determined by optimization (best fit), where the impedance of target tissue (Z1) is known.

The electrical impedance model (FIG. 3A) is converted to a non-target model (FIG. 3B) for non-target tissue by setting the impedance of target tissue (Z1) to zero in the electrical impedance model (FIG. 3A).

The non-target tissue model (FIG. 3B) is used to estimate a non-target component, and the non-target component is removed from an experimental electrical impedance signal to estimate a target signal. The removal occurs in time domain or frequency domain.

The non-target tissue model (FIG. 3B) is used to estimate a non-target signal and the non-target signal is subtracted from an experimental signal to estimate a target signal.

The in vivo target signal can be analyzed to identify and position a spatial electrical impedance variation that represents tumor tissue.

The in vivo target signal can be analyzed to image the tumor tissue, such as on 2D, or 3D EIM images.

In some examples, a computer program 406 comprises computer code that when executed by one or more processors performs one or more of the methods described.

In some examples, an imaging system comprises a controller or other means for performing one or more of the methods described.

Referring back to FIG. 4.

At block 42, 44, 46:

Use in vitro samples to determine the impedance parameter Z1 of a mixed model (Z1, Z2, Z3) which includes target tissue (Z1) and non-target tissue (Z2, Z3).

Obtain Z values for: connective tissue, adipose tissue, fibroadenoma, ductal carcinoma lobular carcinoma and DCIS.

The impedance for target tissue (fibroadenoma or ductal carcinoma or lobular carcinoma or DCIS) is used in the modelling process as Z1.

Z2 and Z3 are connective tissue and adipose tissue and the relative ratios are determined by optimization (best fit).

At block 42:

Collect EIS data with following types of tissues:

TABLE 1
Types of tissues collected by EIS

| | |
|---|---|
| 1. | Pure adipose tissue |
| 2. | Pure connective tissue |
| 3. | Pure infiltrate ductal carcinoma (IDC) |
| 4. | Pure a benign for example, fibroadenoma |
| 5. | IDC mixed with adipose and/or connective tissue |
| 6. | Fibroadenoma mixed with adipose and/or connective tissue |

At block 44, 46: determine Z1 as IDC or fibroadenoma, and Z2 and Z3 as adipose or connective.

At blocks 48, 50, 52: For a particular sample, the associated mixed model (Z1, Z2, Z3) is determined by best fit to in vitro data for the sample (finding Z2, Z3 for the model, Z1 known) e.g., find the optimization parameter R.

Images on the pathology slides of the tissues, are inspected and used to verify how reliable this “best fitted” ratio R is.

At step 54:

The optimized mixed model (Z1, Z2, Z3 e.g., FIG. 3A) is converted to a non-target model for non-target tissue (Z2, Z3, Z1->0, e.g., FIG. 3B).

At block 56:

The non-target tissue model (Z2, Z3; e.g., FIG. 3B) is used to estimate a non-target signal. The non-target signal is subtracted from an experimental signal to estimate a target signal (Z1).

The experimental signal can be an in vitro signal 43 or can be a Range of Interest (ROI) in vivo signal 55.

Analysis of the in vitro impedance data 43 to identify presence of a malignancy such as IDC. In this case, the experimental signal is associated with the particular sample.

Spatial variation of the impedance of the target signal is indicative of IDC in the sample.

Analysis of in vivo data 55 for identification and positioning of the spatial variation AZ that represents a carcinoma or other targeted tissue. In this case, the experimental signal is an in vivo signal 55. Spatial variation of the impedance of the target signal is indicative of a carcinoma in vivo.

Repeat the method blocks 40 to 54 to get appropriate non-target tissue models for different combination of BMI and age.

The composition of tissue will be similar for the particular sample and for the in vivo subject.

The ratio R between Z2 and Z3 can be categorized statistically by different age groups or BMI groups. Which means, since Z1 is a reference impedance, it doesn't change in any classification, however the values of Z2 and Z3 have different range identified by R when we classify them, for example by different age and/or BMI. The non-target tissue model (FIGH 3B) is made appropriate for use with an experimental in vivo signal by look up by age/BMI or other classification parameters.

The non-target tissue model is then used at block 56 to obtain a better data signal 57 for subsequent analysis e.g., imaging.

FIG. 5 illustrates an example of a controller 400. Implementation of a controller 400 may be as controller circuitry. The controller 400 may be implemented in hardware alone, have certain aspects in software including firmware alone or can be a combination of hardware and software (including firmware).

As illustrated in FIG. 5 the controller 400 may be implemented using instructions that enable hardware functionality, for example, by using executable instructions of a computer program 406 in a general-purpose or special-purpose processor 402 that may be stored on a computer readable storage medium (disk, memory etc) to be executed by such a processor 402.

The processor 402 is configured to read from and write to the memory 404. The processor 402 may also comprise an output interface via which data and/or commands are output by the processor 402 and an input interface via which data and/or commands are input to the processor 402.

The memory 404 stores a computer program 406 comprising computer program instructions (computer program code) that controls the operation of the apparatus 10 when loaded into the processor 402. The computer program instructions, of the computer program 406, provide the logic and routines that enables the apparatus to perform the methods illustrated in Figs. The processor 402 by reading the memory 404 is able to load and execute the computer program 406.

The apparatus 10 therefore comprises:
at least one processor 402; and
at least one memory 404 including computer program code
the at least one memory 404 and the computer program code configured to, with the
at least one processor 402, cause the apparatus 10 at least to perform: any one or more of the methods described.

As illustrated in FIG. 6, the computer program 406 may arrive at the apparatus 10 via any suitable delivery mechanism 408. The delivery mechanism 408 may be, for example, a machine readable medium, a computer-readable medium, a non-transitory computer-readable storage medium, a computer program product, a memory device, a record medium such as a Compact Disc Read-Only Memory (CD-ROM) or a Digital Versatile Disc (DVD) or a solid-state memory, an article of manufacture that comprises or tangibly embodies the computer program 406. The delivery mechanism may be a signal configured to reliably transfer the computer program 406. The apparatus 10 may propagate or transmit the computer program 406 as a computer data signal.

Computer program instructions for causing an apparatus to perform at least the following or for performing any one or more of the methods described.

The computer program instructions may be comprised in a computer program, a non-transitory computer readable medium, a computer program product, a machine readable medium. In some but not necessarily all examples, the computer program instructions may be distributed over more than one computer program.

Although the memory 404 is illustrated as a single component/circuitry it may be implemented as one or more separate components/circuitry some or all of which may be integrated/removable and/or may provide permanent/semi-permanent/dynamic/cached storage.

Although the processor 402 is illustrated as a single component/circuitry it may be implemented as one or more separate components/circuitry some or all of which may be integrated/removable. The processor 402 may be a single core or multi-core processor.

References to ‘computer-readable storage medium’, ‘computer program product’, ‘tangibly embodied computer program’ etc. or a ‘controller’, ‘computer’, ‘processor’ etc. should be understood to encompass not only computers having different architectures such as single/multi-processor architectures and sequential (Von Neumann)/parallel architectures but also specialized circuits such as field-programmable gate arrays (FPGA), application specific circuits (ASIC), signal processing devices and other processing circuitry. References to computer program, instructions, code etc. should be understood to encompass software for a programmable processor or firmware such as, for example, the programmable content of a hardware device whether instructions for a processor, or configuration settings for a fixed-function device, gate array or programmable logic device etc.

The blocks illustrated in the Figs may represent steps in a method and/or sections of code in the computer program 406. The illustration of a particular order to the blocks does not necessarily imply that there is a required or preferred order for the blocks and the order and arrangement of the block may be varied. Furthermore, it may be possible for some blocks to be omitted.

Where a structural feature has been described, it may be replaced by means for performing one or more of the functions of the structural feature whether that function or those functions are explicitly or implicitly described.

The systems, apparatus, methods and computer programs may use machine learning which can include statistical learning. Machine learning is a field of computer science that gives computers the ability to learn without being explicitly programmed. The computer learns from experience E with respect to some class of tasks T and performance measure P if its performance at tasks in T, as measured by P, improves with experience E. The computer can often learn from prior training data to make predictions on future data. Machine learning includes wholly or partially supervised learning and wholly or partially unsupervised learning. It may enable discrete outputs (for example classification, clustering) and continuous outputs (for example regression). Machine learning may for example be implemented using different approaches such as cost function minimization, artificial neural networks, support vector machines and Bayesian networks for example. Cost function minimization may, for example, be used in linear and polynomial regression and K-means clustering. Artificial neural networks, for example with one or more hidden layers, model complex relationship between input vectors and output vectors. Support vector machines may be used for supervised learning. A Bayesian network is a directed acyclic graph that represents the conditional independence of a number of random variables.

The term ‘comprise’ is used in this document with an inclusive not an exclusive meaning. That is any reference to X comprising Y indicates that X may comprise only one Y or may comprise more than one Y. If it is intended to use ‘comprise’ with an exclusive meaning then it will be made clear in the context by referring to “comprising only one.” or by using “consisting”.

In this description, reference has been made to various examples. The description of features or functions in relation to an example indicates that those features or functions are present in that example. The use of the term ‘example’ or ‘for example’ or ‘can’ or ‘may’ in the text denotes, whether explicitly stated or not, that such features or functions are present in at least the described example, whether described as an example or not, and that they can be, but are not necessarily, present in some of or all other examples. Thus ‘example’, ‘for example’, ‘can’ or ‘may’ refers to a particular instance in a class of examples. A property of the instance can be a property of only that instance or a property of the class or a property of a sub-class of the class that includes some but not all of the instances in the class. It is therefore implicitly disclosed that a feature described with reference to one example but not with reference to another example, can where possible be used in that other example as part of a working combination but does not necessarily have to be used in that other example.

Although examples have been described in the preceding paragraphs with reference to various examples, it should be appreciated that modifications to the examples given can be made without departing from the scope of the claims.

Features described in the preceding description may be used in combinations other than the combinations explicitly described above.

Although functions have been described with reference to certain features, those functions may be performable by other features whether described or not.

Although features have been described with reference to certain examples, those features may also be present in other examples whether described or not.

The term ‘a’ or ‘the’ is used in this document with an inclusive not an exclusive meaning. That is any reference to X comprising a/the Y indicates that X may comprise only one Y or may comprise more than one Y unless the context clearly indicates the contrary. If it is intended to use ‘a’ or ‘the’ with an exclusive meaning then it will be made clear in the context. In some circumstances the use of ‘at least one’ or ‘one or more’ may be used to emphasis an inclusive meaning but the absence of these terms should not be taken to infer any exclusive meaning.

The presence of a feature (or combination of features) in a claim is a reference to that feature or (combination of features) itself and also to features that achieve substantially the same technical effect (equivalent features). The equivalent features include, for example, features that are variants and achieve substantially the same result in substantially the same way. The equivalent features include, for example, features that perform substantially the same function, in substantially the same way to achieve substantially the same result.

In this description, reference has been made to various examples using adjectives or adjectival phrases to describe characteristics of the examples. Such a description of a characteristic in relation to an example indicates that the characteristic is present in some examples exactly as described and is present in other examples substantially as described.

Whilst endeavoring in the foregoing specification to draw attention to those features believed to be of importance it should be understood that the Applicant may seek protection via the claims in respect of any patentable feature or combination of features hereinbefore referred to and/or shown in the drawings whether or not emphasis has been placed thereon.

1. A method comprising using a non-target tissue model to virtually purify an experimental electrical impedance signal with respect to target tissue.

2. A method as claimed in claim 1, wherein the target tissue is tumor tissue.

3. A method comprising
obtaining a purifying impedance transfer function based on an electrical impedance model with one or more non-target tissue components and without a target tissue component; and
purifying an experimental electrical impedance signal using the purifying impedance transfer function to obtain a virtually purified signal for the target tissue component.

4. A method as claimed in claim 3 comprising: purifying an experimental electrical impedance signal using the purifying impedance transfer function to obtain a virtually purified signal for the target tissue component by assuming an electrical impedance model comprising the target tissue component and the one or more non-target tissue components, wherein the target tissue is tumor tissue and the non-target tissue is non-tumor tissue.

5. A method comprising:
in dependence upon age and/or a measure of obesity, accessing an impedance transfer function dependent upon non-tumor tissue; and
modifying an experimental electrical impedance signal using the obtained impedance transfer function to obtain a signal less dependent upon non-tumor tissue and more dependent upon tumor tissue impedance.

6. A method as claimed in claim 5, wherein the measure of obesity is body-mass-index.

7. A method as claimed in claim 5, wherein the non-tumor tissue impedance is based on connective tissue impedance and adipose tissue impedance.

8. A method as claimed in claim 5, wherein the tumor tissue impedance is a benign such as fibroadenoma tissue impedance or a carcinoma such as IDC tissue impedance.

9. A method as claimed in claim 5, wherein the experimental signal is an electrical impedance signal from an in vitro measurement.

10. A method as claimed in claim 5, wherein the experimental signal is an electrical impedance signal from an in vivo measurement.

11. A method as claimed in claim 5, wherein an electrical impedance signal is used for electrical impedance imaging.

12. A method as claimed in claim 5, wherein the electrical impedance model is a mixed impedance model which includes a target tissue impedance (Z1) and non-target tissue impedance (Z2, Z3).

13. A method as claimed in claim 12, where target tissue impedance Z1 and a first non-target tissue impedance Z3 are in electrical series as a combination, and that combination is in electrical parallel to a second non-target tissue impedance Z2.

14. A method as claimed in claim 13, wherein the target tissue impedance Z1 is one of benign such as fibroadenoma impedance or a carcinoma, such as IDC impedance depending upon a desired target, the first non-target tissue impedance Z3 is one of connective tissue impedance and adipose tissue impedance and the second non-target tissue impedance Z2 is the other one of connective tissue impedance and adipose tissue impedance.

15. A method as claimed in claim 5 comprising using in vitro samples to determine the impedance of target tissue (Z1) and/or using in vitro samples to determine the impedance of non-target tissue, optionally using in vitro samples to determine the first non-target tissue impedance Z3 and/or using in vitro samples to determine the impedance of the second non-target tissue impedance Z2.

16. A method as claimed in claim 5, wherein the electrical impedance model is determined by best fit to in vitro data for one or more samples.

17. A method as claimed in claim 13, wherein a relative ratio of first non-target tissue impedance Z3 to second non-target tissue impedance for the electrical impedance model is determined by optimization (best fit), where the impedance of target tissue (Z1) is known or from purified tissue database, which determined and calibrated from pathological identified cellular based tissue properties.

18. A method as claimed in claim 5, wherein the electrical impedance model is converted to a non-target model for non-target tissue by setting the impedance of target tissue (Z1) to zero in the electrical impedance model.

19. A method as claimed in claim 5, wherein the non-target tissue model (Z2, Z3) is used to estimate a non-target component, and the non-target component is removed from an experimental electrical impedance signal to estimate a target signal, wherein the removal occurs in time domain or frequency domain.

20. A method as claimed in claim 5, wherein the non-target tissue model (Z2, Z3) is used to estimate a non-target signal and the non-target signal is subtracted from an experimental signal to estimate a target signal.

21. A method as claimed in claim 20 comprising analysis of an in vivo target signal to identify and position a spatial electrical impedance variation that represents tumor tissue.

22. A method as claimed in claim 20, comprising imaging the tumor tissue.

23-25. (canceled)
